(19) United States
(12) Patent Application Publication
(10) Pub. No.: US 2014/0020691 A1
(43) Pub. Date: Jan. 23, 2014

(54) ORAL APPLIANCE FOR TREATMENT OF MEDICAL CONDITIONS SUCH AS OBSTRUCTIVE SLEEP APNEA AND SNORING AND FOR IMPROVING ATHLETIC PERFORMANCE AND METHOD OF OPTIMIZING SAME

(71) Applicant: Rest Assured Technologies, Oakmont, PA (US)

(72) Inventors: James E. Sweeney, Pittsburgh, PA (US); James G. Sweeney, Oakmont, PA (US); John Micco, New Castle, PA (US)

(21) Appl. No.: 13/945,850

(22) Filed: Jul. 18, 2013

Related U.S. Application Data
(60) Provisional application No. 61/672,989, filed on Jul. 18, 2012.

(51) Int. Cl. A61F 5/56

(52) U.S. Cl. CPC A61F 5/566
USPC 128/848

(57) ABSTRACT

An oral appliance for treatment of medical conditions, such as obstructive sleep apnea and snoring, and/or for improving athletic performance, comprises a maxillary and a mandibular baseplate component each configured to conform to at least some of the teeth of the user; at least one pair of straps adjustably coupling the baseplate components, wherein the strap defines the relative horizontal positioning of the baseplate components; at least one pair of shim holding brackets coupled to one baseplate component; and a plurality of shims, wherein each shim is replaceably received in the shim holding bracket and configured to abut against the other baseplate component, wherein the shim when received within the shim holding bracket defines the relative vertical positioning of the baseplate components, and wherein the shims are provided in varying heights to allow for varying the vertical positioning of the oral appliance for a specific user.

### CROSS-REFERENCE TO RELATED APPLICATION

This application claims the benefit of U.S. Provisional Patent Application Ser. No. 61/672,989, entitled “RestQ”, filed on Jul. 18, 2012, and which application is incorporated herein by reference.

### BACKGROUND OF THE INVENTION

1. Field of the Invention

The present invention relates generally to an oral appliance for the treatment of medical conditions such as obstructive sleep apnea and snoring and for improving athletic performance and to a method of optimizing same.

2. Background

One of the most widespread sleep disorders is snoring, a common affliction that affects tens of millions of people worldwide, and can potentially have very serious health and social consequences. Snoring is a sound produced by the vibration of tissue caused by a breathing obstruction during sleep. There are many factors associated with snoring including, but not limited to: heredity, body weight, age, gender, smoking history, nasal, and tonsil, or soft palate deformities, use of alcohol, allergies, and sleep position. Commonly, both the snorer and the snorer's sleep partner lose sleep due to the snorer's snoring. The sleep partner is awakened by the sounds caused by snoring, perhaps repeatedly each night. Movement by the sleep partner in subsequently attempting to fall back asleep may then awaken the snorer, if the noise of their own snoring has not already done so. Lack of sleep subsequently leads to daytime fatigue, a compromised immune system, poor mental and emotional health, irritability and lack of productivity, which can lead to further repercussions. Snoring is thus problematic for many people.

Obstructive Sleep Apnea (“OSA”) is a potentially lethal sleep and breathing disorder defined as the cessation of breathing for 10 seconds or more (an apnea) at least five times per hour of sleep. In severe cases, individuals can wake up 300 times per night. When breathing is interrupted by an obstruction in the airway, the body reacts by waking enough to start breathing again. Episodes may occur hundreds of time each night, and may not fully awaken the individual, who remains otherwise unaware of the loud snoring, choking and gasping for air typically associated with OSA. The health risks of sleep apnea include higher risks or occurrences of hypertension, heart attack, stroke, daytime somnolence, depression, fibromyalgia, cardiac arrhythmia, inefficient metabolism, loss of short term memory, weight gain, gastric reflux, high blood pressure, diabetes, severe anxiety, memory and concentration impairment, morning headache, intellectual deterioration, mood swings/temperamental behavior, insomnia, impotence

Although not all snorers have OSA, snoring is a prime indicator of OSA. Many OSA sufferers do not receive a sufficient amount of sleep due to repeated apnea events and arousals which act to prevent REM and deep stage sleep, which can lead to chronic daytime exhaustion and long-term cardiovascular stress. OSA is a cause of an estimated 40,000 deaths each year. An estimated 40 million Americans suffer from some degree of OSA, yet less than 5% are currently undergoing any type of treatment.

The pathogenesis of OSA involves a combination of reduced upper airway size and altered upper airway muscle activity, which causes oral tissue to collapse, and hence a blockage to occur. Other factors which are thought to contribute to OSA include tongue size, soft palate volume, a retro-gnathic mandible, an anterio-posterior discrepancy between the maxilla and the mandible, and obesity.

Snoring and OSA are often associated as generally both are caused by blockage of the pharyngeal airway by, for example, excess tissue when various muscles of the body, including the tongue, relax. As the tongue relaxes, it moves posteriorly, blocking the breathing airway. When the breathing airway is blocked, exhaled air is forced through the airway with increased velocity thereby causing vibration of the soft palette and uvula and tongue, tissue, or other obstruction, thereby creating noise. Snoring is caused by the partial obstruction of breathing during sleep while OSA occurs when the tongue and soft palate collapse onto the back of the throat and completely block the pharyngeal airway, thereby stopping breathing during sleep and restricting the flow of essential oxygen. Thus, a correlation between snoring and OSA is generally recognized in the medical community.

Many attempts have been made to reduce or eliminate snoring and OSA in individuals. Various types of surgery, including tracheostomy, surgery of the soft palate and oropharynx, and reconstructive surgery have been utilized in the treatment of snoring and OSA. Invasive surgery however is costly and not without risk, which effectively eliminates this modality as a truly viable solution to snoring and OSA for the general population. Indeed, many practitioners and patients alike would generally seek to avoid surgical intervention and would welcome a minimally invasive route.

Respiratory therapies for the treatment of OSA have been utilized as well, generally utilizing a ventilator type mask on the subject that supplies air/oxygen at a higher than atmospheric pressures, generally known as Constant Positive Air Pressure systems or CPAP systems. Variations of this therapy have been developed such as what are known as Bi-level Positive Air Pressure systems or BiPAP systems that attempt to reduce pressure during subject exhalation. Other variations are known as Multiple Positive Air Pressure, or MPAP, which again have variable pressure. These systems have a relatively large level of “non-compliance” by the subjects who find it difficult to become accustomed to the mask and associated stress associated with breathing and sleeping in the masks. Noncompliance is the polite way of noting the patient's often hate the treatment so much that they elect to stop the treatment, and treatments that are discontinued are naturally quite ineffective.

It has been recognized that opening or enlarging an airway that may be constricted due to collapse of tissue about the oropharynx will normalize the airflow to the lungs and in doing so will have a direct effect of resolving snoring and OSA as well as diminishing pressure fluctuations in the esophagus which have been associated with GERD. Known in the art are devices (heretofore cumbersome or ineffective) which are designed to restrict the velocity of the ingress and egress of air with the goal of reducing or eliminating snoring. It is generally concluded that by opening the aperture of the airway and maintaining the same, the consequences of a collapsed airway are avoided. When the airway is collapsed, the velocity of air flow increases and causes the vibration of the tongue, soft palate, and other tissue present in the pharyngeal wall during sleep. Thus, when expanded and maintained, such as by the invention herein, the airflow is also maintained and snoring and OSA are minimized or eliminated.

As surgical solutions and CPAP and BiPAP solutions are not practical or desired for all patients, other solutions have been pursued. One class of solutions that has a number of advantages is an oral appliance that the patients wear to reposition the patient's jaw or otherwise maintain the patient's airway. These devices are, generally, less cumbersome than an entire ventilation system such as encountered with CPAP and BiPAP systems.

The patent literature evidences a substantial amount of effort in improving oral appliances for treating OSA. The following is a detailed overview of the patent literature in this area which evidence the work and level of skill in this art. These patents and patent publications are incorporated herein by reference and provide sufficient detail to evidence how those of ordinary skill in the art construct the common elements of the present invention.

U.S. Patent Application Publication No. 2007-0292819 entitled “Oral appliance for treatment of snoring and sleep apnea” This publication discloses an oral appliance which is provided for the treatment of snoring and obstructive sleep apnea and which includes an upper tray adaptable to conform to a user's maxillary dentition and a plurality of lower trays, each of the lower trays adaptable to conform to the user's mandibular dentition. Each of the lower trays is structured to impart a different fixed amount of mandibular advancement.

U.S. Patent Application Publication No. 2007-0209666 entitled “Mandible positioning devices” discloses a pharyngeal airway adjuster, or mandible positioning device, which has a maxillary dentition engagement component and a mandibular dentition engagement component. An adjustable connection couples the maxillary dentition engagement component with the mandibular dentition engagement component. The adjustable connection has a first adjustment screw having a longitudinal axis parallel to the plane and a second adjustment screw having a longitudinal axis perpendicular to the plane. The first and second adjustable screws are independently adjustable and structured to effect horizontal and vertical displacement, respectively, of the maxillary dentition engagement component relative to the mandibular dentition engagement component.

U.S. Patent Application Publication No. 2007-0163603 entitled “Tongue retention device and method of use” discloses a tongue retention device for alleviating sleep apnea and other sleep maladies which has a tooth-anchoring piece that is positioned against the teeth and a tab having at least one aperture for the acceptance of a tongue attachment device.

U.S. Patent Application Publication No. 2007-0079833 entitled “Intraoral mandibular advancement device for treatment of sleep disorders, including snoring, obstructive sleep apnea, and gastro-esophageal reflux disease and method for delivering the same” discloses an intraoral mandibular advancement device to treat problems associated with sleep disorders in a user having an obstructed airway. The device has a main body for attachment to the user's mouth and having a central portion; a protrusive element distending from the central portion of the main body such that when worn by the user the element causes mandibular advancement sufficient to expand the “oropharangeal space” and reduce the obstruction; and a retainer extending from the main body for retention of the device in the user's mouth when worn during the user's sleep state.

U.S. Patent Application Publication No. 2006-0196512, and U.S. Pat. No. 6,845,774, both entitled “Dental device” disclose a fastener suitable for connecting two parts of a dental splint together in a prearranged or predetermined spatial relationship to treat a person suffering from a sleep disorder. In one form the fastener comprises a base member and a cover member arranged to define a cavity therebetween. A movable connector is located within the cavity, the movable connector interconnecting the two parts of the dental splint. The fastener has an adjustor movably connected to the connector such that the position of the connector is moved in response to movement of the adjustor by adjusting the position of the adjustor, the connector in the form of a hook moves the other part of the dental splint so as to reposition the jaws of a person being treated for the sleep disorder.

U.S. Patent Application Publication No. 2005-0236003 entitled “Apnea nipple and oral airway and mandibular advancement device” discloses a sleep apnea prevention device which is designed to move the lower jaw forward, keep teeth and lips apart, and guarantee full oxygenation needs with oral airway that is centered in an anterior dental-buccal space shield and wing portion. This, with mouth guard for lower teeth, is all a unit as a single piece of molded plastic or any other material.

U.S. Patent Application Publication No. 2005-0150504 entitled “Method and device for addressing sleep apnea and related breathing disorders” discloses a device has upper and lower tooth-contacting members and an airway defined between them.

U.S. Pat. No. 8,312,884 entitled “Intra-oral apnea and snoring prevention appliance” discloses an intra-oral device used for people that snore or have sleep apnea which is designed to move a user's lower jaw forward relative to the user's immovable upper jaw and to prevent the user's mouth from inadvertently opening while sleeping. The device includes upper and lower appliances fitted to the users respective upper and lower dental arches. The lower appliance possesses a partially embedded bracket. An adjustment element is integrated into the upper portion of the bracket.

U.S. Pat. No. 6,536,439 entitled “Apparatus and methods for treatment of conditions including obstructive sleep apnea and snoring” discloses a rapid maxillary expansion device which is fitted to teeth of the upper jaw, wherein by operation of the jack screw, the maxilla is expanded.

U.S. Pat. No. 6,516,805 entitled “Apparatus for prevention of snoring and improved breathing during sleep” discloses a dental device in which an upper arch and a lower arch are inserted in a users mouth. A deformable material is included with upper arch and lower arch so as to allow a user teeth molding. A post extends from the upper arch and contacts the lower arch so as to extend the users lower jaw forward, thereby reducing snoring.

U.S. Pat. Nos. 6,374,824, 6,325,064 and 6,305,376 each disclose a device for improving the breathing of a user includes an upper arch adapted to receive at least some of the users upper teeth, a lower arch adapted to receive at least some of the user's lower teeth, a connector adjustably coupling the lower arch to the upper arch, a support surface positioned forwardly, relative to the lower arch, and a tensile member which adjustably couples to the connector and to the lower arch.

U.S. Pat. No. 6,170,485 entitled “Anti-snoring device” discloses an anti-snoring device including a “detachably attachable” upper and lower trays selectively fitting over a person's upper and lower teeth. The trays are formed of a rigid plastic outer shell and include an inner thermoplastic moldable portion. Tongues are integrally formed with the rigid outer shells and extend from each of the upper and lower trays forwardly from the central portions of the upper and lower trays. The tongues are selectively detachably attachable to one another in a plurality of positions and for shifting the lower tray and jaw slightly forward from its normal position. Depressions are formed in the lower and upper trays which align and form breathing openings between the upper and lower trays on each side of the trays central portions.

U.S. Pat. No. 5,983,892 entitled “Device for improving breathing” discloses a device for improving breathing is provided in which an upper arch adapted to receive at least some of a user's upper teeth and a lower arch adapted to receive at least some of the user's lower teeth are adjustably coupled by a connector. The connector is operable to allow lateral motion of the lower arch relative to the upper arch, and to adjustably position the lower arch forwardly relative to the upper arch. The upper arch is adjustably coupled by a connecting apparatus to a face mask that is associated with a gas supplying system.

U.S. Pat. No. 5,954,048 entitled “Device for improving breathing” discloses a device for improving the breathing of a user which includes an upper arch adapted to receive at least some of the users upper teeth and a lower arch adapted to receive at least some of the user's lower teeth. A hook is coupled to the upper arch and is uncoupled from the lower arch until the device is inserted into the users mouth. The hook removably engages the lower arch after the device has been inserted into the user's mouth to adjustably position the lower arch forwardly relative to the upper arch. At least a portion of an adjustor associated with the upper arch may rotate to adjust the hook forwardly relative to the upper arch.

U.S. Pat. No. 5,829,441 entitled “Customizable dental device for snoring and sleep apnea treatment” discloses a mandible extension dental device which includes adjustable upper and lower arch trays and coupling means designed to pull the lower jaw of a user forwardly during sleep, so as to minimize snoring and mild apnea conditions. Each of the arch trays includes a substantially rigid synthetic body equipped with a pair of generally L-shaped, continuous adjustment slots. Thermoplastic molding material within each tray permits custom fitting of the trays to the patient.

U.S. Pat. No. 5,755,219 entitled “Device for improving breathing” discloses a device for improving breathing which includes an upper arch adapted to receive at least some of a users upper teeth and a lower arch adapted to receive at least some of the user's lower teeth. A tensile member is coupled to the upper arch and the lower arch, and exerts a tensile force upon the lower arch. The device adjustably positions the lower arch forwardly, relative to the upper arch, using the tensile force.

Work in this field earlier than the above mentioned U.S. Patents and U.S. Patent Application Publications include the oral appliances shown and described in U.S. Pat. Nos. 5,720,302, 5,590,643, 5,566,683, 5,537,994, 5,427,117, 5,409,017, 5,316,020, 5,117,816, 5,092,346, 5,056,534, 5,003,994, 4,901,737, 4,715,368, 4,304,227, 4,169,473, and 3,434,470. See also U.S. Design Pat. No. D685,098 entitled “Sleep apnea appliance”. The above mentioned U.S. Patents and U.S. Patent Application Publications give an overview of the work in the art and help to define the level of skill in the art, the problems to be solved and the prior solutions. These are incorporated herein by reference.

These above described oral appliances and similar oral appliances, however, often have adjustment mechanisms that are difficult to operate. Additionally, known oral appliances incorporating adjustment mechanisms tend to be bulky, thereby adversely impacting the user's comfort level. Furthermore, known oral appliances are difficult to properly fit and may not allow sufficient vertical or horizontal movement of the jaw when worn.

There remains a need for a cost effective oral appliance for the treatment of sleep disorders which is easy to manufacture and properly adjust which is minimally invasive to the patient. It is an object of the present invention to develop an oral appliance for treatment of a medical condition such as obstructive sleep apnea and snoring that overcomes the drawbacks of the above cited prior art.

Proper jaw positioning through the use of oral appliances has expanded to the field of improving athletic performance. Researchers in this aspect of oral appliance applications, such as Dr. Anil Makkar, have noted that when an athlete's jaw is down and forward, the back of the athlete's neck aligns with the back of the athlete's spine, and when the athlete has total alignment the athlete exhibits increased strength, balance, range of motion, and an increase in flexibility and endurance. Dr. Anil Makkar founded Makkar Athletics Group Inc, dedicated to the principles of neuromuscular science and whose flagship product, the MAKKAR PPM™, launched in 2006, is a sports appliance that is custom-made to attempt to align the jaw to its optimal resting position. All-Pro NFL Athletes, PGA & LPGA Golfers, NBA All-Stars, MLB Batting Champs, as well as World Champion boxers and mixed martial artists (MMA) have used such sports appliances to improve athletic performance.

The advantages of the oral appliance for treating medical conditions of the present invention can be easily expanded to improving athletic performance and incorporated into an oral sports appliance and associated method for fitting such a sports appliance.

### SUMMARY OF THE INVENTION

The present invention provides an oral appliance for treatment of medical conditions, such as obstructive sleep apnea and snoring, and/or for improving athletic performance.

In accordance with one aspect of the invention, an oral appliance for treatment of medical conditions, such as obstructive sleep apnea and snoring, and/or for improving athletic performance, comprises a maxillary and a mandibular baseplate component each configured to conform to at least some of the teeth of the user; at least one strap on each side adjustably coupling the baseplate components, wherein the straps defines the relative horizontal positioning of the baseplate components; at least one shim holding bracket on each side coupled to one baseplate component; and a plurality of shims, wherein each shim is replaceably received in the shim holding bracket and configured to abut against the other baseplate component, wherein the shim when received within the shim holding bracket defines the relative vertical positioning of the baseplate components, and wherein the shims are provided in varying heights to allow for varying the vertical positioning of the oral appliance for a specific user.

In accordance with one aspect of the invention, an oral appliance for treatment of medical conditions, such as obstructive sleep apnea and snoring, and/or for improving athletic performance, said oral appliance comprises a maxillary baseplate component configured to conform to at least some of the teeth of the user; a mandibular baseplate component configured to conform to at least some of the teeth of the user; a pair of straps, each strap adjustably coupling the maxillary baseplate component to the mandibular baseplate component, wherein the straps define the relative horizontal positioning of the baseplate components; a pair of shim holding brackets coupled to maxillary baseplate component; and a plurality of pairs of shims, wherein each shim of a pair of shims are replaceably received in one shim holding bracket and configured to abut against the mandibular baseplate component, wherein each pair of shims when received within the shim holding brackets define the relative vertical positioning of the baseplate components, and wherein the plurality pairs of shims are provided in varying heights to allow for varying the vertical positioning of the oral appliance for a specific user.

In accordance with one aspect of the invention, a method of optimizing an oral appliance for treatment of medical conditions, such as obstructive sleep apnea and snoring, and/or for improving athletic performance, said optimizing comprising the steps of: a) Providing an oral appliance including a maxillary baseplate component, a mandibular baseplate component, at least one pair of straps adjustably coupling the maxillary baseplate component to the mandibular baseplate component, at least one pair of shim holding brackets coupled to one baseplate component, and a plurality of shims, wherein each shim is replaceably received in the shim holding bracket and configured to abut against the other baseplate component, b) Conforming the mandibular baseplate component to at least some of the teeth of the patient; c) Conforming the maxillary baseplate component to at least some of the teeth of the patient; d) Positioning at least one shim within an associated shim holding bracket to define the relative vertical positioning of the baseplate components; e) Positioning the at least one pair of straps to define the relative horizontal positioning of the baseplate components; f) Evaluating the relative mandibular placement with the oral appliance; g) replacing the shim within each associated shim holding bracket with a shim of distinct height to define another relative vertical positioning of the baseplate components; h) Evaluating the relative mandibular placement with the oral appliance another relative vertical positioning of the baseplate components and selecting shims for optimizing the oral appliance for the patient.

These and other advantages of the present invention will be clarified in the detailed description of the preferred embodiments taken together with the attached figures wherein like reference numerals reference like elements throughout.

### BRIEF DESCRIPTION OF THE DRAWINGS

FIG. 1 schematically illustrates a perspective view of an oral appliance for treatment of medical conditions, such as obstructive sleep apnea and snoring, and/or for improving athletic performance in accordance with one aspect of the present invention;

FIG. 2 schematically illustrates a perspective view of the oral appliance of FIG. 1 illustrating the relative mandibular positioning with the oral appliance in accordance with one aspect of the present invention;

FIG. 3 schematically illustrates a side elevation view of the oral appliance of FIG. 1;

FIG. 4 schematically illustrates an exploded perspective view of the oral appliance of FIG. 1;

FIG. 5 schematically illustrates a perspective view a modified mandibular baseplate component of the oral appliance of FIG. 1;

FIGS. 6-8 are schematic perspective views of the maxillary baseplate component of the oral appliance of FIG. 1;

FIG. 9a schematically illustrates a perspective view of a shim vertical adjustment assembly of the oral appliance of FIG. 1;

FIG. 9b schematically illustrates side elevation views of distinct shims for the vertical adjustment assembly of FIG. 9a; and

FIG. 10 schematically illustrates perspective views of the individual components attached to the baseplates of the oral appliance of FIG. 1.

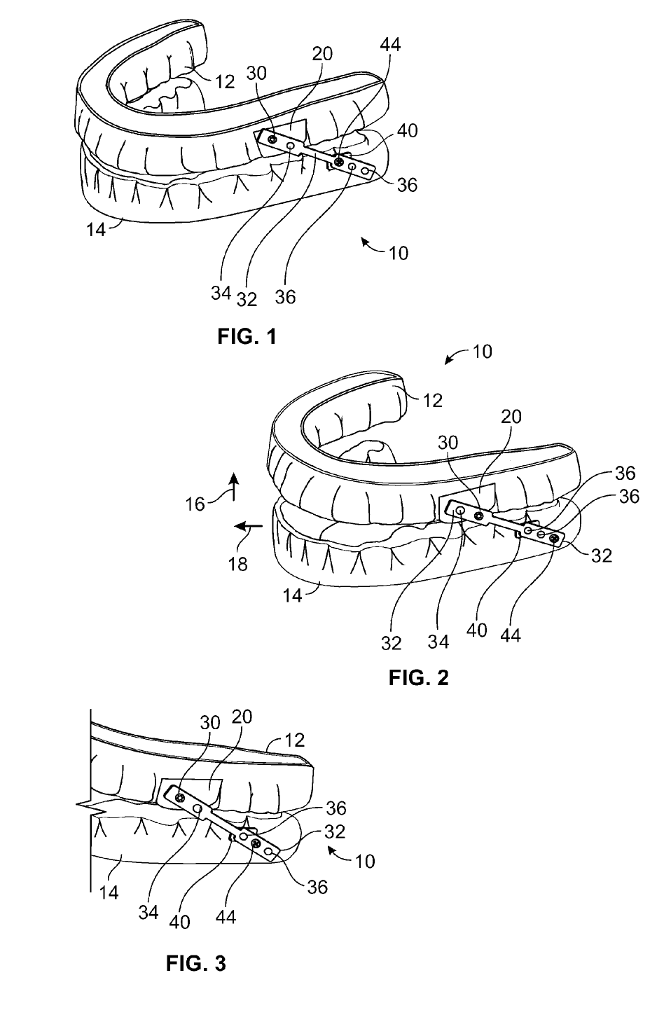

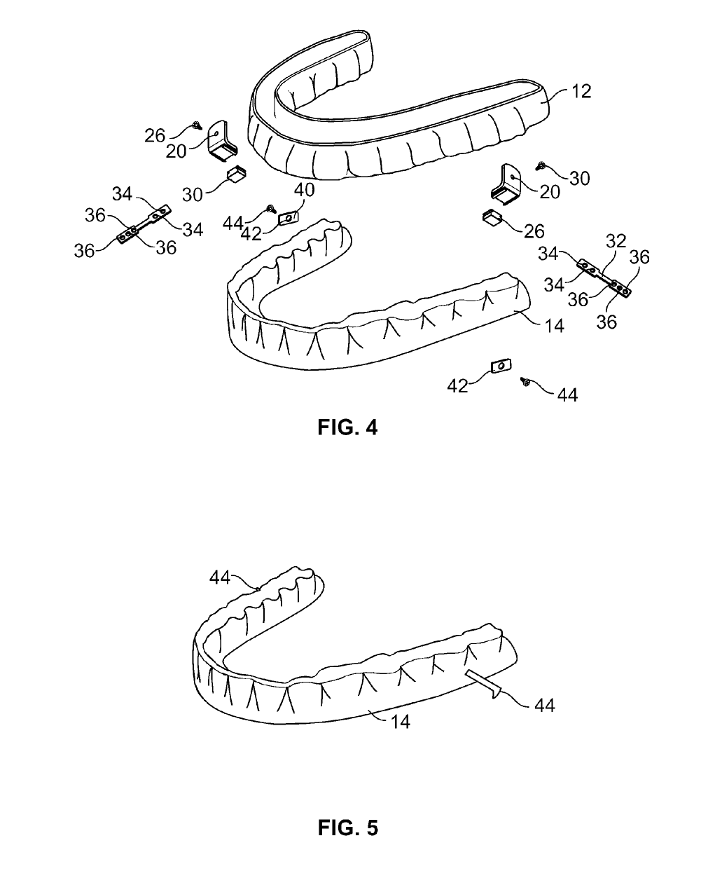

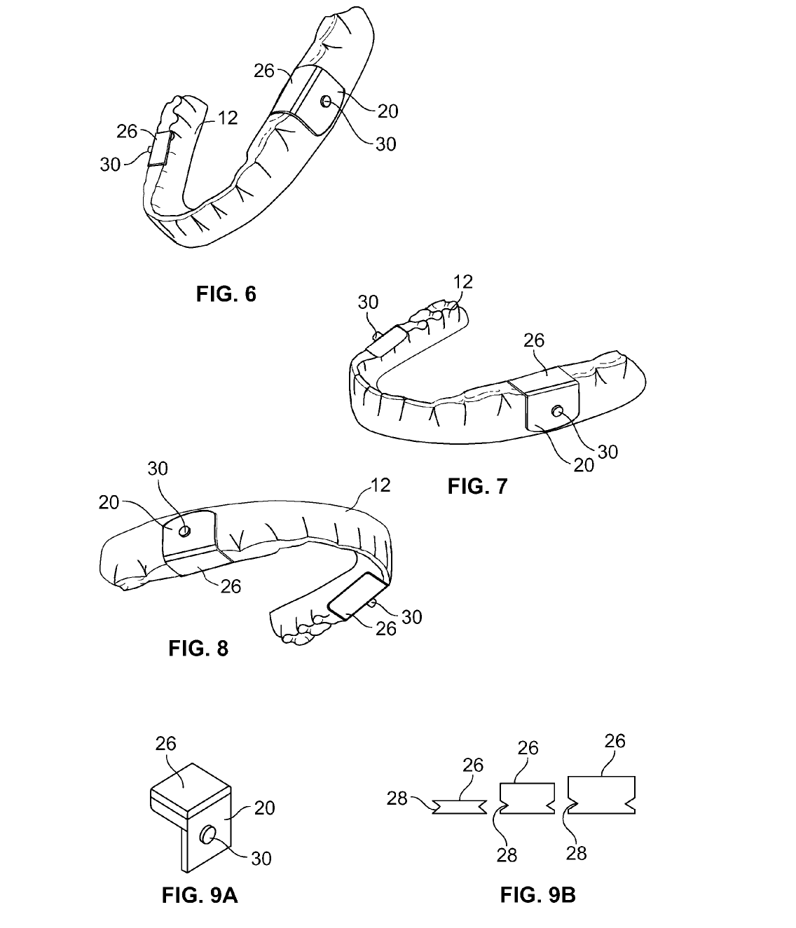

### DETAILED DESCRIPTION OF THE INVENTION

It is noted that, as used in this specification and the appended claims, the singular forms “a,” “an,” and “the” include plural referents unless expressly and unequivocally limited to one referent. The various embodiments and examples of the present invention as presented herein are each understood to be non-limiting with respect to the scope of the invention.

The present invention provides an oral appliance 10 for treatment of medical conditions, such as obstructive sleep apnea and snoring, and/or for improving athletic performance. The oral appliance 10 comprises a maxillary baseplate component 12 configured to conform to at least some of the teeth of the patient and a mandibular baseplate component 14 configured to conform to at least some of the teeth of the user. The oral appliance 10 described below will allow dentists to prepare and optimize an appliance for a patient in their labs without multiple appliance re-molding and without a large adjustment mechanism being present in the appliance. The patient may be an athlete where the present appliance is being used to improve athletic performance; the patient may also be referenced as the subject or user of the appliance 10. The oral appliance 10 will allow dentists to adjust the vertical position and horizontal position of the mandible as schematically represented by arrows 16 and 18, as will be clarified in the following description.

The molding/conforming of the baseplate components 12 and 14 to the user and the materials for forming this structure are well known to those of ordinary skill in the art. The simplification of the adjustable vertical and horizontal positioning with the oral appliance 10 of the present invention allows for baseplate components 12 and 14 to be formed of conventional molding material, rather than a more complex outer shell and inner molding components. The oral appliance 10 could, in theory, also be formed with a hard outer shell and molded inner component, as some prior art structure, but need not be. The final determination is a consideration of cost and user acceptance. A two component baseplate (inner and outer shell) will be disfavored if it unduly increases the bulk of the appliance 10 where user's become uncomfortable, or if it unduly increases the costs. A simple one piece molded construction for the baseplate components 12 and 14 is shown.

The amount of material and size/length of each baseplate components 12 and 14 is mainly determined by the amount of dentation support required to effectively adjust the mandible position of the user. Using too small of an anchor point (short baseplate component) can place an undue stress on the user's existing dentation. The size is generally known in the art. Additionally the baseplate components 12 and 14 may include additional material, such as when necessary for forming a sports appliance of the present invention that is used simultaneously as a mouth guard, such as may be used in boxing or MMA events.

A key aspect of the present invention is the provision of a pair of shim holding brackets 20 coupled to maxillary baseplate component 12. The brackets 20 may be held by adhesives, bonded, or press fit in and thermally bonded with the conforming procedure. Alternative attaching methods may be utilized. Each bracket 20 includes a threaded opening 22 and a dovetail engaging member 24 on a top surface thereof opposed form the mandibular baseplate component 14. The brackets 20 may be formed of metal or hard plastic.

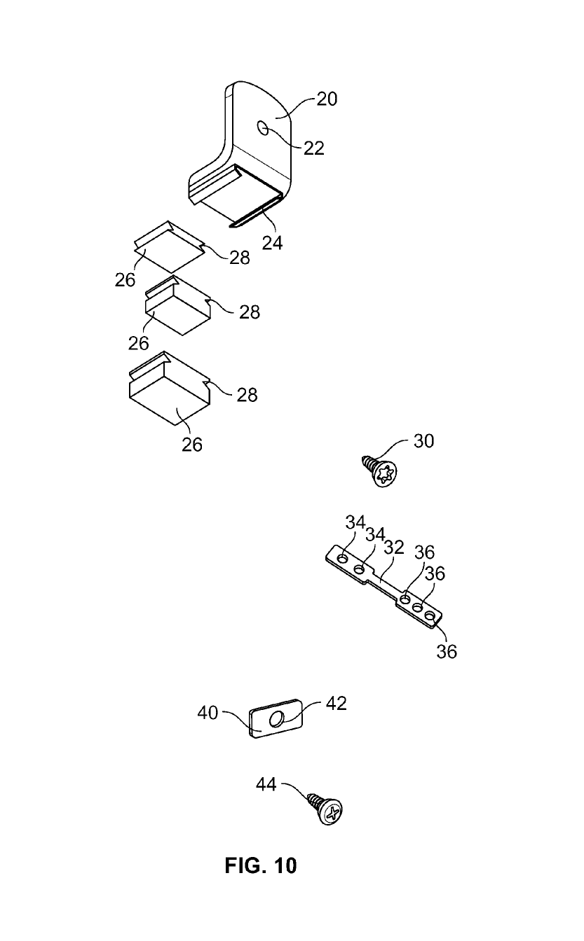

Operating with the pair of brackets 20 are a plurality of pairs of shims 26. Each shim 26 is easily formed as a substantially rectangular member extending substantially the width of the baseplate components 12 and 14 to form a sufficiently large bearing surface to properly position the baseplate components 12 and 14. The shims 26, like the brackets 20, may be efficiently formed of metal or hard plastic. Each shim 26 includes dovetail notches 28 corresponding in relative position to the dovetail engaging members 24 on a top surface of the brackets 20. Each of the shims 26 of a pair of shims 26 are replaceably received in one shim holding bracket 20 through engagement of the notches 28 with members 24 and configured to abut against the mandibular baseplate component 14. Each pair of shims 26, when received within the shim holding brackets 26, define the relative vertical positioning of the baseplate components 12 and 14. Friction or a low shear strength adhesive may be used to help maintain the shims 26 in position in a manner that they can be easily replaced. The plurality pairs of shims 26 are provided in varying heights, as shown best in FIGS. 9b and 10, to allow for varying the vertical positioning of the oral appliance 10 for a specific user. A number of pairs of shims 26 may be provided, the set of three is merely for illustration and not to be restrictive of the invention.

The oral appliance 10 according to the invention includes a strap retaining member coupled to each bracket 20, such as a strap retaining screw 30 threaded into hole 22 on each bracket. The screw 30 could be replaced with pins glued into openings or other fastening members that would be known to those of ordinary skill in the art. The screw 30 represents a simple construction for this component. The oral appliance 20 further includes a pair of straps 32, each strap 32 adjustably coupling the maxillary baseplate component 12 to the mandibular baseplate component 14. The straps 32 define the relative horizontal positioning of the baseplate components 12 and 14 and may also be easily formed out of metal or hard plastic. The straps 32 are flexible but not elastic thereby defining the horizontal position.

Each strap 32 includes a plurality of positions, or openings 34, to engage the strap retaining member, retaining screw 30, on each shim holding bracket 20. Each strap 32 includes a plurality of positions, or openings 36, to engage an opposing strap retaining member, such as a retaining screw 44, coupled to the mandibular baseplate component 14. A pair of attachment plates or brackets 40 may be coupled to the mandibular baseplate component 14. The brackets 40 may be bonded, held by adhesives, or press fit in and thermally bonded with the conforming procedure. Alternative attaching methods may be utilized. Each bracket 40 includes a threaded opening 42 receiving the retaining screw 44. The brackets 44 and screw 42 may also be formed of metal or hard plastic.

The result of the above described components is a cost effective efficient oral appliance for treatment of medical conditions, such as obstructive sleep apnea and snoring, and/or for improving athletic performance, which can be adjusted and implemented easily by a dentist with his own lab.

In practice, for a patient who may be suffering from a sleep disorder that could be treated with the appliance 10 of the present invention the initial step is evaluating the patient for a risk of a sleep disorder by examining the presence and level of various risk factors, such as snoring, high blood pressure, partner exhibiting symptoms of lack of sleep, waking up gasping, headaches in the morning, tired during the day, neck size, tongue size, crowding in the pharynx, body mass index, mandible position. The quantification of these risk factors will indicate the possibility that the appliance 10 of the present invention will be appropriate for the patient.

In a preferred implementation of the present invention, patients who have been evaluated and identified as having a high level or sever risk of sleep disorders may be forwarded for a clinical diagnosis that may involve a home sleep study and/or a lab sleep study by qualified sleep physician or practitioner. A resulting diagnosis will typically result in an appropriate treatment modality such as a life style change, surgery, CPAP/MPAP/BiPAP treatment, Oral Appliance (10), and combinations thereof.

For those identified as benefitting from an oral appliance a dental exam is conducted to assure they are a candidate for the implementation of the oral appliance 10. This can include a review if the patient has enough dentation to support and utilize the appliance 10 without undue stress on the remaining teeth and if other physical constraints prevent use of the appliance 10.

For those patients for which the appliance 10 is suitable the next step is optimizing the oral appliance 10 generally comprising the steps of: a) Providing an oral appliance 10 including a maxillary baseplate component 12, a mandibular baseplate component 14, at least one pair of straps 32 adjustably coupling the maxillary baseplate component 12 to the mandibular baseplate component 14, at least one pair of shim holding brackets 20 coupled to one baseplate component 12, and a plurality of shims 26, wherein each shim 26 is replaceably received in the shim holding bracket 20 and configured to abut against the other baseplate component 14, b) Conforming the mandibular baseplate component 12 to at least some of the teeth of the patient; c) Conforming the maxillary baseplate 14 component to at least some of the teeth of the patient; d) Positioning at least one shim 26 within an associated shim holding bracket 20 to define the relative vertical positioning of the baseplate components 12 and 14; e) Positioning the at least one pair of straps 32 to define the relative horizontal positioning of the baseplate components 12 and 14; f) Evaluating the relative mandibular placement with the oral appliance 10; g) replacing the shim 26 within each associated shim holding bracket 20 with a shim 26 of distinct height to define another relative vertical positioning of the baseplate components 12 and 14; h) Evaluating the relative mandibular placement with the oral appliance another relative vertical positioning of the baseplate components and selecting shims for optimizing the oral appliance for the patient. This optimizing step can be referenced as finding the sweet spot for the vertical and horizontal position of the mandible via the appliance 10, and these steps can be easily accomplished in the dentist's office with the appliance 10 of the invention.

For some patients the actual sweet spot may require slow adjustments over time. In other words the patient begins with shims 26 less than the total height of the final sweet spot until the patient gets accustomed to this size and is ready to move up to the next height of shim 26. The easy construction of the shimming vertical adjustment of the appliance 10 can allow the patient to be given the various pairs of shims and to self adjust the steps at their own pace.

Additionally some patients may go through another sleep study to evaluate the results and adjust treatments as necessary.

As mentioned, the present invention is also well suited for enhancing sports performance due to neuromuscular factors and improvements in respiration/airflow. The determination of the “sweet spot” in such applications can involve measurements of select muscle group capacity at various vertical and horizontal positions. The sports appliance applications may further require adding “mouth guard” aspects. For example the size of the appliance may be larger to accommodate the “mouthguard” functions, then would strictly be necessary for mandibular positioning aspects, but this would not change the relevant portions of the invention described herein. It should be known to those in the art that the sports appliance of the present invention would be used generally only during play of the sport, as opposed to the medical appliance worn typically at night.

The present invention has been described with reference to specific details of particular embodiments thereof. It is not intended that such details be regarded as limitations upon the scope of the invention except insofar as and to the extent that they are included in the accompanying claims. A number of variations to the present invention will be apparent to those of ordinary skill in the art and these variations will not depart from the spirit and scope of the present invention. For example, the brackets 20 could be used on the mandibular or maxillary base plates 12 and 14 and the vertical adjustment provided by brackets 20 and shims 26 can be implemented on other oral appliances using bilateral—horizontal adjustment. The scope of the invention is defined by the appended claims and equivalents thereto.

We claim:

1. An oral appliance for treatment of medical conditions, such as obstructive sleep apnea and snoring, and/or for improving athletic performance, said oral appliance comprising:
a maxillary baseplate component configured to conform to at least some of the teeth of the user;
a mandibular baseplate component configured to conform to at least some of the teeth of the user;
at least one pair of straps adjustably coupling the maxillary baseplate component to the mandibular baseplate component, wherein the strap defines the relative horizontal positioning of the baseplate components;
at least one pair of shim holding brackets coupled to one baseplate component; and
a plurality of shims, wherein each shim is replaceably received in the shim holding bracket and configured to abut against the other baseplate component, wherein the shim when received within the shim holding bracket defines the relative vertical positioning of the baseplate components, and wherein the shims are provided in varying heights to allow for varying the vertical positioning of the oral appliance for a specific user.

2. The oral appliance according to claim 1 further including a single pair of shim holding brackets.

3. The oral appliance according to claim 2 wherein each shim is a substantially rectangular member extending substantially the width of the baseplate components.

4. The oral appliance according to claim 3 further including a dovetail engagement structure between each shim and an associated shim holding bracket.

5. The oral appliance according to claim 1 further including a strap retaining member on each shim holding bracket, wherein each strap is coupled to one of the baseplate components through the strap retaining member on the shim holding bracket.

6. The oral appliance according to claim 5 further including an opposing strap retaining member coupled to the baseplate component not including the shim holding bracket, wherein each strap is coupled to this baseplate component through the opposing strap retaining member.

7. The oral appliance according to claim 6 further including a plurality of positions on each strap to engage the strap retaining member on each shim holding bracket.

8. The oral appliance according to claim 7 further including a plurality of positions on each strap to engage the opposing mandibular strap retaining bracket.

9. An oral appliance for treatment of medical conditions, such as obstructive sleep apnea and snoring, and/or for improving athletic performance, said oral appliance comprising:
a maxillary baseplate component configured to conform to at least some of the teeth of the user;
a mandibular baseplate component configured to conform to at least some of the teeth of the user;
a pair of straps, each strap adjustably coupling the maxillary baseplate component to the mandibular baseplate component, wherein the straps define the relative horizontal positioning of the baseplate components;
a pair of shim holding brackets coupled to maxillary baseplate component; and
a plurality of pairs of shims, wherein each shim of a pair of shims are replaceably received in one shim holding bracket and configured to abut against the mandibular baseplate component, wherein each pair of shims when received within the shim holding brackets define the relative vertical positioning of the baseplate components, and wherein the plurality pairs of shims are provided in varying heights to allow for varying the vertical positioning of the oral appliance for a specific user.

10. The oral appliance according to claim 9 wherein each shim is a substantially rectangular member extending substantially the width of the baseplate components.

11. The oral appliance according to claim 9 further including a dovetail engagement structure between each shim and an associated shim holding bracket.

12. The oral appliance according to claim 9 further including a strap retaining member on each shim holding bracket, wherein each strap is coupled to the maxillary baseplate component through the strap retaining member on the shim holding bracket.

13. The oral appliance according to claim 12 further including an opposing strap retaining member coupled to the mandibular baseplate component, wherein each strap is coupled to mandibular baseplate component through the opposing strap retaining member.

14. The oral appliance according to claim 13 further including a plurality of positions on each strap to engage the strap retaining member on each shim holding bracket.

15. The oral appliance according to claim 14 further including a plurality of positions on each strap to engage the opposing strap retaining member.

16. A method of optimizing an oral appliance for treatment of medical conditions, such as obstructive sleep apnea and snoring, and/or for improving athletic performance, said optimizing comprising the steps of:
a) Providing an oral appliance including a maxillary baseplate component, a mandibular baseplate component, at least one pair of straps adjustably coupling the maxillary baseplate component to the mandibular baseplate component, at least one pair of shim holding brackets coupled to one baseplate component, and a plurality of shims, wherein each shim is replaceably received in the shim holding bracket and configured to abut against the other baseplate component,
b) Conforming the mandibular baseplate component to at least some of the teeth of the patient;
c) Conforming the Maxillary baseplate component to at least some of the teeth of the patient;
d) Positioning at least one shim within an associated shim holding bracket to define the relative vertical positioning of the baseplate components;
e) Positioning the at least one pair of straps to define the relative horizontal positioning of the baseplate components;
f) Evaluating the relative mandibular placement with the oral appliance;
g) replacing the shim within each associated shim holding bracket with a shim of distinct height to define another relative vertical positioning of the baseplate components;
h) Evaluating the relative mandibular placement with the oral appliance another relative vertical positioning of the baseplate components and selecting shims for optimizing the oral appliance for the patient.

17. The method of optimizing an oral appliance according to claim 16 wherein a plurality of horizontal positions are evaluated in optimizing the oral appliance through varying positions on the strap.

18. The method of optimizing an oral appliance according to claim 16 wherein the appliance further includes a strap retaining member on each shim holding bracket, wherein each strap is coupled to the maxillary baseplate component through the strap retaining member on the shim holding bracket.

19. The method of optimizing an oral appliance according to claim 16 wherein the appliance further includes an opposing strap retaining member coupled to the mandibular baseplate component, wherein each strap is coupled to mandibular baseplate component through the opposing strap retaining member.

20. The method of optimizing an oral appliance according to claim 16 wherein each shim is a substantially rectangular member extending substantially the width of the baseplate components.
